(19) United States
(12) Patent Application Publication
(10) Pub. No.: US 2012/0058145 A1
(43) Pub. Date: Mar. 8, 2012

(54) METHOD FOR PRODUCING VIRUS FROM CELL CULTURE INVOLVING HOMOGENIZATION

(76) Inventors: Bruno Rene Andre, Rixensart (BE); Benoit Paul Suzanne Champluvier, Rixensart (BE)

(21) Appl. No.: 13/319,393

(22) PCT Filed: May 6, 2010
(86) PCT No.: PCT/EP2010/056164

§ 371 Date: Nov. 8, 2011

Related U.S. Application Data
(60) Provisional application No. 61/176,562, filed on May 8, 2009.

(51) Int. Cl. A61K 39/145; C07K 14/11; A61P 37/04; A61K 39/12; A61P 31/12; A61P 31/16; C12N 7/02; C12N 7/04

(52) U.S. Cl. 424/210.1; 435/239; 530/396; 435/236; 424/204.1

(57) ABSTRACT

A method of purifying a virus, or a viral antigen thereof, from a culture of infected cells comprising at least the steps of: (a) collecting the virus-containing cell culture medium, and (b) purifying the virus, wherein at least one homogenization step is implemented during the purification to obtain a virus homogenate.

### STATEMENT REGARDING FEDERALLY SPONSORED RESEARCH

Aspects of this invention were made with United States government support pursuant to Contract # HHSO100200600011C, from the Department of Health and Human Services; the United States government may have certain rights in the invention.

### TECHNICAL FIELD

The present invention relates to a method for purifying viruses, or viral antigens, produced by cell culture, to the viruses or viral antigens obtainable by this method and to vaccines which contain such viruses or viral antigens. In particular, the invention provides a method for improving the virus yield.

### TECHNICAL BACKGROUND

Due to the vast number of diseases caused by viruses, virology has been an intensively studied field. There has always been the demand to produce viruses efficiently in order to isolate and purify viral proteins, to generate vaccines, to prepare analytical tools, or to provide viruses for laboratory studies.

Recently, cell culture-based technologies as an alternative to the traditional egg-derived production systems have been developed.

Cell culture systems appear as a suitable alternative mode of vaccine preparation in particular, simpler, flexible, consistent, allowing to improve possibilities of up-scaling vaccine production capacities and thus to reach large quantities of virus, if needed, in particular, in case of a pandemic threat or a terrorist attack.

However, after production, the cell culture-produced virus requires to be recovered from the cell culture, and, when appropriate, to be purified. Various processes of cell culture-produced virus are known in the art, including methods for recovering the virus from the cell culture and for purifying it. Such processes present the major drawback of providing a low virus yield, as virus material is lost along the different steps required for these processes. In particular, aggregation has a negative impact on virus yield and this may affect many different steps in a virus purification process. Therefore, a need remains for providing alternative and, preferably, improved methods for virus recovery and purification from cell culture.

### SUMMARY OF THE INVENTION

The method according to the present invention provides a solution to overcome this drawback, as it is intended for limiting the virus loss during the recovery and purification process, and, therefore, increasing the virus yield. In particular, the present method helps reduce aggregation formation which may be responsible for virus loss during the different steps employed for purifying a virus produced by cell culture.

In a first aspect of the present invention, there is provided a method of recovering a virus, or a viral antigen thereof, produced in cell culture, comprising at least the steps of:
(a) obtaining a fluid comprising the virus, or a viral antigen thereof, and
(b) subjecting the fluid to at least one homogenization step to obtain a virus homogenate

In a second aspect, there is provided a method of purifying a virus, or a viral antigen thereof, from a culture of infected cells, comprising at least the steps of:
(c) collecting the virus-containing cell culture medium, and
(d) purifying the virus,
wherein at least one homogenization step is implemented during the purification to obtain a virus homogenate.

In a third aspect, there is provided a virus, or a viral antigen thereof, obtainable according to the method of the present invention.

In a fourth aspect, there is provided an immunogenic composition comprising a virus, or a viral antigen thereof, obtained according to the method of the present invention.

In a fifth aspect, there is provided a method for the preparation of a vaccine comprising at least the step of admixing the virus obtained according to the present invention with a pharmaceutically acceptable carrier.

### DESCRIPTION OF DRAWINGS

FIG. 1: After harvesting, Influenza virus-containing cell culture medium was homogenized by high-pressure at 700 bars. A sample of harvest was collected before and after homogenization and subjected to a CPS disc centrifuge analysis to determine the size of the particles present within the harvest. Results are presented in the form of a graph. The “no HPH” curve corresponds to the control harvest, where no homogenization has been performed, while the “HPH” curve corresponds to the harvest which has been homogenized.

FIG. 2: After harvesting, Influenza virus-containing cell culture medium was homogenized by high-pressure at different pressures (700, 1000, 1250 and 1500 bars) with two homogenizer devices, Rannie™ and Panda™. The integrity of the virus after homogenization treatments was analysed by sucrose gradient ultracentrifugation. The different fractions, pellet (P), intermediate layers (M) and supernatant (S) were collected after the ultracentrifugation and the presence of the virus was assessed by analysing the HA content in the different fractions by Western-blot analysis with an anti-HA antibody. Samples from lane 2 onwards are successively supernatant (S), intermediate layer (M) and sucrose cushion pellet fractions (P) of untreated control, Rannie™ 700 bars, Rannie™ 1000 bars, Panda™ 700 bars, Panda™ 1000 bars, Panda™ 1250 bars and Panda™ 1500 bars, respectively.

FIG. 3: Shown are images captured via electron microscopy of influenza virus harvests. FIG. 3A represents a non-homogenized harvest, while FIG. 3B represents a harvest which has been homogenized with a Rannie™ device at 700 bars.

### DETAILED DESCRIPTION

The present invention relates to an improved method of recovering and purifying viruses from cell culture that can be applied to both small and large scale virus production. In particular, the method according to the invention helps increase the virus yield by implementing a particular step before any critical step providing a low virus yield and improving that step yield. The present invention presents the advantage that this particular step may be employed as many times as it is desired in a purification process, as the inventors observed that it does not have any detrimental effect on the virus viability and integrity. In particular, the inventors surprisingly observed that implementing a homogenization step after collecting the virus and/or during its purification, prior to specific steps throughout the purification process, resulted in increasing the virus yield obtained after said steps, while the virus integrity is maintained. The additional advantage conferred by the present invention is that homogenization, which is a mechanical procedure, does not rely on adding chemicals, like detergents, or enzymes, which may require their subsequent elimination, depending on what application is intended for the resulting purified virus. For instance, when the purified virus is to be included in a vaccine, such constituents, like detergents or enzymes, should be avoided and highly reduced, when used, for safety and regulatory issues.

Homogenization also has an unexpected positive impact on the amount and/or size of equipment which needs to be used for further purifying the cell culture-produced virus. In particular, the inventors found out that homogenizing a virus-containing fluid before clarifying said fluid by filtration allowed to significantly reduce the filtration surface area required for clarification, as compared to the surface filtration area required for clarifying a virus-containing fluid which has not been previously homogenized. This point is particularly advantageous when proceeding to large scale production of cell culture-produced viruses. This advantage may, in particular, result from a better filterability of the virus-containing fluid which has been homogenized before filtration, compared to a virus-containing fluid with no prior homogenization. According to the present invention, “filterability” is to be understood as the capacity of a fluid to be filtered. Two features may be used for characterizing the filterability of a fluid: (i) the occurrence of filter plugging, indicative of a low filterability, and (ii) the inlet pressure which increases when the filterability is low, as accumulation on the filter starts to plug the filter, and decreases when the filterability is higher. Filterability may thus be defined as the volume of fluid that can pass through the filter before it plugs and can no longer pass.

In the sense of the present invention, “homogenization” is to be understood as a step making a heterogeneous mixture the same throughout the entire substance, so as to render it homogenous. The fluid obtained after homogenization is, thus, called a “homogenate”. Homogenization is, particularly, used for disrupting molecular interactions which are responsible for aggregation occurring within a particles-containing fluid, said particles forming aggregates.

Homogenization of a particles-containing fluid is known to occur, in particular, by subjecting the fluid (i) to shearing by liquid flow, (ii) to exploding by pressure differences between inside and outside of particles, (iii) to collision forces by impact of beads or paddles, or (iv) a combination of theses forces. It is believed that disruption occurs by a combination of spatial pressure and velocity gradients, turbulence, cavitation, impact with solid surfaces and extensional stress. The present invention contemplates, as homogenization step, any mechanical technique which, after its application to a particles-containing fluid, results in the reduction of both the aggregates number and their size within said fluid. Non-limiting examples of homogenization are (i) bead impact methods, where particles-containing fluids are homogenized by throwing beads against the particles. Parameters, such as bead selection (density, diameter and quantity), agitation speed or run duration will be optimized according to the material to be homogenized and to the disruption degree that needs to be achieved; (ii) high-pressure homogenization (HPH), in particular, where the stream of suspended particles is forced at high-pressure down a narrow channel or across the small gap of a valve. This accelerates its speed, thereby stretching and shearing particles. Optionally, the moving stream is subsequently and abruptly impacted against an obstacle to further disintegrate particles. This obstacle may be an impingement wall; (iii) microfluidizer, where the stream of pressurized particles suspension fluid is split into two legs. These are then directed at one another in an interaction chamber where they collide at high velocity, disrupting particles; and (iv) sonication relying on the use of sound waves in fluids for disrupting particles.

The available commercial devices for homogenization allow to handle small to very large volumes of fluid. Some of them require to proceed to batch operations, while others offer the possibility of operating in a continuous mode. As an illustration only, the following devices may be cited: BEAD BEATER™ (from Biospec) and DYNO™-MILL (from GlenMills) for Bead Impact homogenizers, FRENCH™ PRESS (from Thermo Scientific), RANNIE™ and GAULIN™ (from APV), as well as PANDA™ and PANTHER™ (from Niro Soavi GEA Process Engineering Inc) for HPH homogenizers, and SONICATOR 3000™ (from Misonix) for sonication.

According to one embodiment, homogenization step(s) of the method of the present invention is (are) performed by HPH. Alternatively, sonication is used as the homogenization step(s) in the method of the present invention. According to a specific embodiment, homogenization may be performed by HPH and sonication in the method of the present invention.

A high-pressure homogenizer consists of a positive displacement pump, a homogenizing valve and, sometimes, an impingement wall. The pump is used to force the fluid into the homogenizing valve where the work is done. Attached to the pump is a homogenizing valve assembly, typically, consisting of a seat (often called valve seat, bottom part), a valve (top part) and an impact wall or ring, when present, also called impingement wall (Diels and Michiels “High Pressure homogenization as a non-thermal technique for the inactivation of microorganisms”, Critical Reviews in Microbiology, 32: 201-216 and Goldberg “Mechanical/physical methods of cell disruption and tissue homogenization”, Methods in Molecular Biology, vol. 424: 3-22). The unhomogenized fluid is forced under high pressure and low velocity to enter the valve area. In the homogenizing valve, as the fluid enters the adjustable close clearance area between the valve and seat, there is a rapid increase in velocity with a corresponding decrease in pressure and the fluid is accelerated radially. The intense energy release causes turbulence and localised pressure differences which will tear apart particles. The fluid leaves the valve in the form of a radial jet that strikes an impact ring. Finally, the homogenized fluid exits the homogenizer at low velocity and essentially at atmospheric pressure. The operating pressure is controlled by adjusting the distance between the seat and the valve. By adjusting the gap between the valve and the seat, the flow area in the homogenizing valve is controlled. When the flow area is reduced, pressure within the pump discharge manifold increases. When the flow area is increased, the pressure is reduced. The high pressure generated by the pump is converted to fluid velocity and heat as the fluid is discharged from the restricted area in the homogenizing valve. Several parameters may be critical and can be optimized so as to achieve the aggregation disassembly level that is searched for, such as the pressure, the temperature, and/or the number of cycles. Also, one parameter which may affect the homogenization efficiency independently of any of the previous one is the geometry of the valve. Indeed, a few different valves, each with a specific geometry, are available and have been shown to result in different homogenization efficiency. By way of example, the “knife edge” shape valve is known to be particularly effective.

The terms “fluid comprising a virus” and “virus-containing fluid” are synonymous and are to be understood as any liquid preparation comprising a virus, irrespective of its purification status. The fluid may have not been purified at all. For instance, the fluid may be the cell culture supernatant collected after cells were infected with a virus and the virus was replicated and released into the medium, or the cells infected with the virus themselves, or both. Alternatively, the fluid may have been partially purified. For example, the virus-containing fluid may have been pre-clarified, by filtration or by centrifugation, before being subjected to at least one homogenization step. In one embodiment of the present invention, homogenization is performed on the virus-containing cell culture medium. In an alternative embodiment, the virus-containing cell culture medium is pre-clarified before being subjected to homogenization. In the sense of the present invention, pre-clarification means that a rough clarification is performed to eliminate large complexes before homogenization and that a further clarification will be performed on homogenized virus-containing fluid to eliminate contaminants of smaller size. For instance, pre-clarification on a 5 μm membrane filter may be performed on the virus-containing cell culture medium before homogenization, said homogenization being followed by further clarification on a 0.2 μm membrane filter. Therefore, according to one embodiment, the invention provides for a method of recovering a virus, or a viral antigen thereof, produced in cell culture, comprising the steps of (a) obtaining a fluid comprising the virus or viral antigen thereof, and (b) subjecting the fluid to a least one homogenization step to produce a virus homogenate. According to another embodiment, the invention provides for a method of purifying a virus, or viral antigen thereof, produced by cell culture, comprising at least the steps of: (c) collecting the virus-containing cell culture medium, and (d) purifying the virus, wherein at least one step of homogenization is implemented during the purification to obtain a virus homogenate. In a specific embodiment, the fluid comprising a virus, in particular, the virus-containing cell culture medium is successively homogenized by HPH or sonication and clarified by filtration or centrifugation. According to an alternative embodiment, the virus-containing cell culture medium is successively pre-clarified by filtration or centrifugation, homogenized by HPH, and further clarified by filtration.

Accordingly, a “fluid comprising a virus” in the sense of the present invention may comprise intact cells, cell debris and/or other cell constituents, such as soluble proteins and DNA, additionally to the virus. Indeed, intact cells and cell debris may be floating in the cell culture medium after infection and the intracellular content of cells may be released into the medium, due to spontaneous cell lysis after virus infection. Viruses are either in a free form, i.e. detached from cells and/or cell debris, or cell-associated, i.e. attached to cells and/or cell debris. Free viruses may be in the form of aggregates, as viruses tend to aggregate, or in the form of individual particles, i.e. non-aggregated. This is the case, in particular, of influenza viruses for which the aggregation phenomenon is documented.

Aggregation, which may be an intrinsic property of a virus, may also be favoured, as a general, non-specific, event under certain circumstances. For instance, the concentration of a virus within a fluid, may lead, eventually, to non-specific aggregates formation, as increasing the number of particles within a given volume will favour, and increase the risk of random collisions between said particles. Temperature may also have an influence on aggregation. The presence of certain constituents in the virus environment, i.e. included in the virus-containing fluid, may, as well, be accountable for causing virus aggregation. As specific examples of such constituents, with respect to Influenza viruses, may calcium and magnesium be cited as compounds inducing influenza virus aggregation, when present in a virus preparation. Some host cell proteins, involved in helping virus replication and assembly within the cells, which may have, possibly, be released into the medium due to spontaneous cell lysis, may also be responsible for inducing virus aggregation when present in a virus-containing fluid. Virus purification processes are complex processes relying on multiple steps. Some of these steps may require the presence of constituents favouring aggregation, but used for a distinct purpose essential in the purification path. For instance, nucleases for degrading nucleic acids from host cells present in the virus-containing fluid are used, as the elimination of such nucleic acids may be highly desirable, in particular, when the purified virus is to be included in a vaccine. These enzymes often need elements, such as magnesium, for their activity. Therefore, a nucleic acid degradation step using a nuclease may induce aggregation. A distinct example of a compound used in a virus purification process for its ability to inactivate the virus, but known to be responsible for aggregation is the formaldehyde. It may be, thus, advantageous to perform a homogenization step after a step of formaldehyde treatment. Similarly, ultrafiltration is frequently used to concentrate a virus early in the purification process, so as to reduce the volume to be purified, and rendering thus the subsequent manipulation of the volume, during the rest of the purification process, easier. As indicated above, aggregation may be promoted by such a concentration step.

In the sense of the present invention, “aggregation” is to be understood as the formation of cells/cells, cells/viruses, and viruses/viruses complexes of varying size. The term “cells” should not be restricted to the cell as such. It encompasses intact cells, cell debris or cell fragments, as well as cell constituents present inside the cells and which have been released, in particular, due to possible spontaneous lysis of the cells after their infection with a virus. By way of examples, may DNA, RNA and soluble proteins be cited as host cells elements which may be present within a virus-containing fluid according to the present invention.

The method according to the present invention, by implementing homogenization step(s), aims at reducing aggregates size and/or number, providing, thus, more free viruses, and, advantageously, more free viruses in a non-aggregated form. Interestingly, the method according to the invention can be applied to any type of aggregation, irrespective of the type of interaction which is responsible for aggregates formation and of the strength of the interactions. The method of the present invention may also be used regardless of the type of aggregation which is involved, whether it is a specific property of the virus or whether it is due to random collisions as described above.

Homogenization may be performed before any suitable step of a virus purification process. In particular, homogenization should be performed before any step which is negatively affected by the presence of aggregation, in that said aggregation is responsible for virus loss during that step.

Homogenization may also be performed after any step, whose conditions favoured aggregates formation, whether it is virus/virus aggregates or any type of cell/virus aggregates, as described earlier. Cell/virus aggregates would result in trapping a virus population which may, as a consequence, be eliminated with cell contaminants, when purifying the virus from the virus-containing fluid, if said viruses are not previously dissociated from cells or cell debris. A similar issue is raised with virus/virus aggregates, as large aggregates, depending on their size, may also be lost during purification, even though viruses are in a free form, i.e. detached from cells or cell debris. For example, if purification occurs through the elimination of contaminants based on their bigger size or density, compared to viral particles size or density, then, virus aggregates above a certain size or density, will also be eliminated. Such a principle is, in particular, applicable to any step involving filtration on filters or membranes with a certain nominal porosity, or centrifugation, including density gradient ultracentrifugation, which steps are frequently used during virus purification processes.

In order to identify which step, in a virus purification process, leads to aggregates formation and to what extent, it is possible to monitor the aggregation level over the different steps of a process, by measuring, for instance, the size of particles present within a sample collected at any time of a process, such as, for example, before and after a particular step. Any known-in-the-art technique may be used. Aggregation monitoring may be performed by a visual analysis, using optic or electron microscopy images, which allows to visualize the aggregates in a direct manner, if any, and evaluate their global size, as well as the numbers of aggregates units, i.e. the number of individual components forming the aggregates. If needed, such an analysis may be associated with real size calculation. Techniques based on differential sedimentation allowing to separate different populations according to their respective size or density may also be used. More recent approaches are based on dynamic light scattering, also called photo correlation spectroscopy, and are also suitable for evaluating aggregation within a sample to be tested. Any technique allowing to discriminate between individuals particles and larger aggregates is suitable for assessing the aggregation level within a sample, whether of a direct type or indirect type. As a non-limiting example may the CPS disc centrifuge analysis be cited as a method for measuring particles size. The technique will be chosen depending on the expected size of individual particles, for instance the non-aggregated virus of interest, and/or on the expected size difference between the different populations to be analysed, as the sensitivity of the techniques may vary.

These techniques are also useful for determining the homogenization efficiency after a fluid has been homogenized. Indeed, the same techniques can be used for analysing the particles size within a sample, before and after its homogenization, so as to observe a reduction in large aggregates population with a concomitant increase in smaller size populations, reflecting the homogenizing effect.

When appropriate, these techniques should be calibrated with positive controls, such as, for instance, testing and analysing a purified solution of the virus of interest.

Homogenization techniques and appropriate conditions will be chosen and optimized according to the type of virus and the type of cells used for the virus production. Conditions should be adapted to the aggregation level that is present in the fluid to be homogenized and to the aggregates dissociating level that is searched for. Aggregates may be more or less disassembled, depending on the strength of the homogenization which is applied to the virus-containing fluid. For instance, if high-pressure homogenization is used, then varying the applied pressure will have an impact on the aggregation disassembly level. In the sense of the present invention, the term “high-pressure (HPH)” means a pressure higher than about 50 bars, in particular, higher than about 100 bars, more particularly, higher than about 500 bars. In one embodiment of the present invention, the pressure applied during a HPH step ranges from 100-1500 bars, in particular, 500-1200, and more particularly, 700-1000. In a specific embodiment, when homogenization is performed by HPH, the pressure is about 700 bars. Depending on the cells and/or viruses, interactions responsible for aggregation formation within a fluid may have a varying strength. Therefore, some aggregates may be more resistant to homogenization, and will require more stringent conditions. As indicated above, varying the pressure may help improve the homogenization, or disruption efficiency. The number of cycles may also have a positive impact on aggregation disruption efficiency and this number will be optimized according to specific experiments. The present invention contemplates at least one cycle of homogenization per homogenization step, suitably, at least two cycles, and, more suitably, at least 3 cycles per homogenization step. With regard to HPH, the homogenizing valve geometry should also be a parameter to be taken into account, when optimizing the HPH conditions.

According to the results of the particles size analysis techniques, any given step in a virus purification process may then be targeted for a prior homogenization so as to increase the virus yield of this particular step. The homogenization may also be implemented after a targeted step, so a to increase the virus yield of the subsequent step.

For example, when performing a clarification step, a homogenization step is suitably carried out before clarification to avoid that viruses trapped in large cells or cell debris complexes be lost as being retained on the filter when clarification is made by filtration or be lost in the sedimented pellet when clarification occurs by centrifugation. As a consequence, prior homogenization of a virus-containing fluid before clarifying said fluid will improve the virus yield obtained after clarification. Accordingly, in one embodiment of the method of the present invention, homogenization is implemented prior to clarification of a virus-containing fluid. In a specific embodiment, clarification is performed by filtration. In a distinct embodiment, clarification is performed by centrifugation. Optionally, prior to said homogenization, i.e. the homogenization performed before clarification, the virus-containing fluid may be pre-clarified so as to eliminate large contaminants before proceeding to homogenization. The positive effect of prior homogenization on the virus clarification yield is not restricted to specific filters. Any type of filters may be used for clarification after homogenization. Suitable ones may use cellulose filters, regenerated cellulose filters, cellulose fibers combined with inorganic filter aids and organic resins, or any combination thereof and polymeric filters. Although not required, a multiple filtration process may be carried out, like a two- or three-stage process consisting, for instance, in sequentially and progressively removing impurities according to their size, using filters with appropriate nominal pore size, in particular, filters with decreasing nominal pore size, allowing to start removing large precipitates and cell debris. In addition, single stage operations employing a relatively tight filter or centrifugation may also be used for clarification. More generally, any clarification approach including, but not limited to, dead-end filtration, depth filtration, microfiltration, or centrifugation, which provide a filtrate of suitable clarity to not foul the membrane and/or resins in subsequent steps, will be acceptable to use in the clarification step of the present invention. Accordingly, according to a specific embodiment of the present invention, the viral clarification step is performed by depth filtration, in particular but not exclusively using a three-stage filtration train composed, for example, of three different depth filters with nominal porosities of 5 μm-0.5 μm-0.2 μm. In a specific embodiment according to the present invention, the virus-containing cell culture medium is successively homogenized by HPH and clarified with a 5 μm-0.5 μm-0.2 μm filtration train. In an alternative embodiment, the virus-containing fluid, such as the virus-containing cell culture medium, is successively pre-clarified on a 5 μm filter membrane, homogenized, for example by HPH, and further clarified on a 0.2 μm filter membrane. In a distinct embodiment, the virus-containing fluid, such as the virus-containing cell culture medium, is successively homogenized, by HPH for instance, clarified by centrifugation, and optionally further clarified by depth filtration, for example, using a 0.2 μm filter.

If using membranes or filters which are not neutral but positively charged, it may be useful to implement an additional step of rinsing said membrane or filter with a rinsing buffer comprising salts to elute the virus fraction which may have been retained due to ionic interactions with the membrane or filter. One example of suitable salt which may be included in the rinsing buffer is sodium chloride (NaCl), which may be present at a concentration ranging from 0.1M to 2M, in particular, from 0.5M to 1.5M, suitably 1M. In one embodiment of the invention, when clarification is performed by membrane filtration, whether it is pre-clarification or clarification, said clarification comprises a membrane rinsing step with a buffer comprising NaCl, in particular, NaCl 1M.

When clarifying by filtration, a prior homogenization step, by reducing the size and number of aggregates of any type, presents the additional and simultaneous advantage of limiting the filter surface area required for clarifying the virus-containing fluid, as mentioned earlier. It is particularly advantageous when doing large scale production. Indeed, due to the reduction in aggregates formation, globally resulting in a fluid comprising particles of smaller size, the risk of plugging the membrane because of the presence of large aggregates is decreased. Depending on the extent to which aggregates are disrupted, i.e. on the final particles size achieved after homogenization, and on the porosity of the membrane filter used for clarification, a decreased risk may mean that membrane plugging will not be completely prevented, but will be, at least, delayed, in the sense that a bigger volume will be filtered before plugging on a given membrane happens, if the fluid has been homogenized first. The plugging occurrence and strength may be evaluated by monitoring, during the filtration operation, the inlet pressure. The more plugged a membrane filter is, the higher the inlet pressure value is. The inventors observed that when homogenizing first a virus-containing fluid before filtering it, then, during subsequent filtration, the inlet pressure value was reduced when compared to a similar fluid which had not been homogenized first, indicating that plugging is, at least, limited, if not prevented. As a consequence, the filtration area for a given volume to be filtered was reduced, as a bigger volume could be filtered on the same membrane, without requiring to change the membrane because of its plugging.

According to the present invention, during the purification of the virus from the cell culture medium, the virus suspension may be subjected to ultrafiltration (sometimes referred to as diafiltration when used for buffer exchange) for concentrating the virus and/or buffer exchange. This step is particularly useful when the virus to be purified is diluted, as is the case when pooling virus harvest collected by perfusion over a few days post-inoculation. The process used to concentrate the virus according to the method of the present invention can include any filtration process where the concentration of virus is increased by forcing diluent to be passed through a filter in such a manner that the diluent is removed from the virus suspension whereas the virus is unable to pass through the filter and thereby remains in a concentrated form in the virus preparation. Ultrafiltration may comprise diafiltration which is an ideal way for removal and exchange of salts, sugars, non-aqueous solvents, removal of material of low molecular weight, of rapid change of ionic and/or pH environments. Microsolutes are removed most efficiently by adding solvent to the solution being ultrafiltered at a rate equal to the ultrafiltration rate. This washes microspecies from the solution at a constant volume, isolating the retained virus. Diafiltration is particularly advantageous when a downstream step requires that a specific buffer be used in order to get an optimal reaction. For example, implementing a diafiltration step before degrading host cell nucleic acids with a nuclease may allow to perform the nuclease reaction in a buffer specific and optimal for that nuclease. Concentration and diafiltration may also be implemented at any suitable step of the purification process, when it is wanted to remove undesirable compounds, such as sucrose, after a sucrose gradient ultracentrifugation, or such as formaldehyde, after a step of virus inactivation with formaldehyde. The system is composed of three distinct process streams: the feed solution (comprising the virus), the permeate and the retentate. Depending on the application, filters with different pore sizes may be used. In the present invention, the retentate contains the virus and can be used for further purification steps, if desired. The membrane composition may be, but is not limited to, regenerated cellulose, polyethersulfone, polysulfone, or derivatives thereof. The membranes can be flat sheets (also called fait screens) or hollow fibers. As described earlier, concentrating a virus, by ultrafiltration for example, may promote non-specific virus aggregation. Therefore, implementing a homogenization step after concentrating a virus fluid is expected to disrupt this aggregation, so that any subsequent step will not be negatively impacted by aggregation. In one embodiment, the method of the invention comprises at least one ultrafiltration/diafiltration step during the virus purification and homogenization is performed after said step. Accordingly, in a specific embodiment of the present invention, the virus-containing fluid, such as the virus-containing cell culture medium, is successively pre-clarified, in particular, on a 5 μm filter or by centrifugation, concentrated by ultrafiltration, homogenized by HPH or sonication, and further clarified, for instance on a 0.2 μm filter.

Depending on what application is the cell culture-produced virus purified for, it may be desirable also to eliminate from the virus suspension host cell nucleic acids contaminants. In particular, when the purified virus is to be included in a vaccine, host cell nucleic acids should be degraded and eliminated from the purified virus. Nucleic acids degradation frequently occurs through the use of nucleases targeting RNA and DNA. A non-limiting example of a suitable nuclease for degrading host cell nucleic acids is Benzonase™. Benzonase™, or any other suitable nuclease, may be added at any suitable step of a virus purification process. Not only may aggregation be induced after treating a virus-containing fluid with a nuclease, but aggregation may negatively impact the nuclease reaction. As discussed earlier, certain enzymes, such as nucleases, including Benzonase™, require that elements such as calcium and/or magnesium be present in the reaction buffer for their activity. However, such elements may also promote virus aggregation formation. As a consequence, virus aggregates may form during the nuclease treatment. In terms of nuclease efficacy, aggregation may also be an issue. It is possible that nucleic acids be complexed and trapped within aggregates of cell, cell debris and/or virus. These trapped nucleic acids would not be accessible to the nuclease and, would, therefore, escape to degradation by the nuclease. With regard to nuclease treatment, the aggregation issue, whether before the treatment or as a consequence of it, can be overcome by implementing a homogenization step at an appropriate moment, respectively, immediately before treating a virus-containing fluid with a nuclease or immediately after said treatment. In one embodiment, the method according to the invention comprises a nuclease degradation step, suitably a Benzonase™ treatment, and homogenization is performed either before adding the nuclease or after the nuclease treatment, or both before and after. For instance, a nuclease may be added to the retentate obtained after ultrafiltration of a clarified virus-containing cell-culture medium, whether said virus-containing cell-culture medium has been already homogenized or not. The present invention also contemplates that homogenization be implemented more than once in a virus purification process. Accordingly, in a specific embodiment of the invention, the virus-containing cell culture medium is successively homogenized, in particular by HPH, clarified by filtration or by centrifugation, concentrated by ultrafiltration, homogenized by HPH, treated with Benzonase™ and, optionally re-homogenized. In an alternative embodiment of the present invention, the virus-containing cell culture medium is successively treated with Benzonase™, homogenized, for instance by HPH, and clarified by filtration or by centrifugation.

If desired, the virus obtained according to the present invention may be further purified using standard techniques employed for virus purification such as density gradient centrifugation, for instance sucrose gradient ultracentrifugation and/or chromatography, such as ion exchange chromatography.

If implementing a density gradient ultracentrifugation step during the virus purification process, it may be advantageous to homogenize the virus-containing fluid before loading it on the centrifuge rotor, as aggregates, if present within the fluid, may negatively impact the ultracentrifugation step yield. As discussed previously, depending on the aggregates size and whether viruses are trapped in aggregates, some virus loss may occur during this type of step. Therefore, aggregation disruption by homogenization, before loading the virus-containing fluid, may help increase the virus yield obtained after ultracentrifugation. Accordingly, in one embodiment, wherein a sucrose gradient ultracentrifugation is implemented to purify a cell culture-produced virus, homogenization of the virus-containing fluid is performed prior to the loading on the centrifuge rotor. Accordingly, in a specific embodiment of the invention, the virus-containing cell culture medium is, successively, homogenized by HPH, clarified by filtration or by centrifugation, concentrated by ultrafiltration, optionally re-homogenized by HPH and subjected to at least one sucrose gradient ultracentrifugation step. In an alternative embodiment, a Benzonase™ treatment step is implemented between the ultrafiltration step and the homogenization step.

The virus prepared according to the present invention can be used for any purpose, including, for instance, purification of viral proteins, analytical assays, infection of host cells, diagnostic purposes or therapeutic or prophylactic uses such as vaccination and clinical administration.

Interestingly, the inventors observed that homogenization does not affect the virus viability. Depending on the virus type, homogenization conditions may require to be optimized so as to maintain the virus viability, as viruses may have different specific resistance to homogenization. Any known-in-the-art method for assessing virus viability can be used for that purpose in the context of the present invention, such as, for instance, an electron microscopy analysis of viral particles, a sucrose gradient centrifugation analysis, or a virus titration allowing to check the infectivity of the virus produced according to the method of the present invention.

The method of the invention is amenable to a wide range of viruses, any virus which is capable of infecting cells and using them for its replication, including, but not limited to, adenoviruses, hepadnaviruses, herpes viruses, orthomyxoviruses, papovaviruses, paramyxoviruses, picornaviruses, poxviruses, reoviruses and retroviruses. In particular, the method of invention is suitable for enveloped viruses, such as myxoviruses. In one embodiment, the viruses produced by the method of the invention belong to the family of orthomyxoviruses, in particular, influenza virus.

Viruses or viral antigens may be derived from an Orthomyxovirus, such as influenza virus. Orthomyxovirus antigens may be selected from one or more of the viral proteins, including hemagglutinin (HA), neuraminidase (NA), nucleoprotein (NP), matrix protein (M1), membrane protein (M2), one or more of the transcriptase (PB1, PB2 and PA). Particularly suitable antigens include HA and NA, the two surface glycoproteins which determine the antigenic specificity of the Influenza subtypes.

The influenza virus can be selected from the group of human influenza virus, avian influenza virus, equine influenza virus, swine influenza virus, feline influenza virus. Influenza virus is more particularly selected in strains A, B and C, preferably from strains A and B.

Influenza antigens may be derived from interpandemic (annual or seasonal) influenza strains. Alternatively, influenza antigens may be derived from strains with the potential to cause a pandemic outbreak (i.e., influenza strains with new hemagglutinin compared to hemagglutinin in currently circulating strains, or influenza strains which are pathogenic in avian subjects and have the potential to be transmitted horizontally in the human population, or influenza strains which are pathogenic to humans). Depending on the particular season and on the nature of the antigen included in the vaccine, the influenza antigens may be derived from one or more of the following hemagglutinin subtypes: H1, H2, H3, H4, H5, H6, H7, H8, H9, H10, H11, H12, H13, H14, H15 or H16. Preferably, the influenza virus or antigens thereof are from H1, H2, H3, H5, H7 or H9 subtypes.

The cells which are used in the method according to the invention can in principle be any desired cell type of cells which can be cultured in cell culture and which can support virus replication. They can be both adherently growing cells or cells growing in suspension. They can be either primary cells or continuous cell lines. Genetically stable cell lines are preferred.

Mammalian cells are particularly suitable, for example, human, hamster, cattle, monkey or dog cells.

A number of mammalian cell lines are known in the art and include PER.C6, HEK cells, human embryonic kidney cells (293 cells), HeLa cells, CHO cells, Vero cells, and MDCK cells.

Suitable monkey cells are, for example, African green monkey cells, such as kidney cells as in the Vero cell line. Suitable dog cells are, for example, kidney cells as in the MDCK cell line.

Suitable mammalian cell lines for growing influenza virus include MDCK cells, Vero cells, or PER.C6 cells. These cell lines are all widely available, for instance, from the American Type Cell Culture (ATCC) collection.

According to a specific embodiment, the method of the invention uses MDCK cells. The original MDCK cell line is available from the ATCC as CCL-34, but derivatives of this cell line may also be used, such as the MDCK cells adapted to growth in suspension (WO 1997/37000.

Alternatively, cell lines for use in the invention may be derived from avian sources, such as chicken, duck, goose, quail or pheasant. Avian cell lines may be derived from a variety of developmental stages including embryonic, chick and adult. In particular, cell lines may be derived from the embryonic cells, such as embryonic fibroblasts, germ cells, or individual organs, including neuronal, brain, retina, kidney, liver, heart, muscle, or extraembryonic tissues and membranes protecting the embryo. Chicken embryo fibroblasts (CEF) may be used. Examples of avian cell lines include avian embryonic stem cells (WO01/85938) and duck retina cells (WO05/042728). In particular, the EB66® cell line derived from duck embryonic stem cells is contemplated in the present invention. Other suitable avian embryonic stem cells include the EBx® cell line derived from chicken embryonic stem cells, EB45, EB14 and EB14-074 (WO2006/108846). This EBx cell line presents the advantage of being a stable cell line whose establishment has been produced naturally and did not require any genetic, chemical or viral modification. These avian cells are particularly suitable for growing influenza viruses.

According to a particular embodiment, the method of the invention uses EB66 cells.

Cell culture conditions (temperature, cell density, pH value, etc . . . ) are variable over a very wide range owing to the suitability of the cells employed and can be adapted to the requirements of particular virus growth conditions details. It is within the skilled in the art person's capabilities to determine the appropriate culture conditions, as cell culture is extensively documented in the art (see, for example, Tissue Culture, Academic Press, Kruse and Paterson, editors (1973), and R. I. Freshney, Culture of animal cells: A manual of basic technique, fourth edition (Wiley-Liss Inc., 2000, ISBN 0-471-34889-9).

In a specific embodiment, host cells used in the method described in the present invention are cultured in serum-free and/or protein-free media. A “serum-free medium” (SFM) means a cell culture medium ready to use that does not require serum addition allowing cell survival and cell growth. This medium is not necessarily chemically defined and may contain hydrolyzates of various origin, from plant for instance. Such serum-free medium present the advantage that contamination with viruses, mycoplasma or unknown infectious agents can be ruled out. “Protein-free” is understood to mean cultures in which multiplication of the cells occurs with exclusion of proteins, growth factors, other protein additives and non-serum proteins. Optionally trypsin or other proteases that may be necessary for viral growth. The cells growing in such culture naturally contain protein themselves.

Serum-free media are commercially available from numerous sources, for instance, VP SFM (Invitrogen Ref 11681-020), Opti-Pro (Invitrogen, Ref 12309-019), or EX-CELL (JHR Bioscience).

Cell may be grown in various ways, for instance, in suspension, or adhering to surfaces, including growth on microcarriers, or combinations thereof. Culturing can be done in dishes, flasks, roller bottles, or in bioreactors, using batch, fed-batch, or continuous systems, such as perfusion systems. Typically, cells are scaled-up from a master or working cell bank vial through various sizes of flasks or roller bottles and finally to bioreactors. In one embodiment, the cells employed according to the method of the invention are cultured on microcarrier beads in a serum-free medium in a stirred-bioreactor and the culture medium is provided by perfusion.

In an alternative embodiment, cells are cultured in suspension in a batch mode.

Prior to infection with the virus, cells are cultured around 37° C., more suitably at 36.5° C., at a pH ranging from 6.7 to 7.8, suitably around 6.8 to 7.5, and more suitably around 7.2

According to the method of the invention, the production of cell culture-based viruses includes generally the steps of inoculating the cultured cells with the viral strain to be produced and cultivating the infected cells for a desired period of time so as to allow virus replication.

In order to produce large quantities of cell-produced viruses, it may be advantageous to inoculate cells with the desired virus strain once cells have reached a high density. Usually, the inoculation is performed when the cell density is at least around 5×10⁶ cells/ml, preferably 6×10⁶ cells/ml, more preferably 7×10⁶ cells/ml, or even higher. The optimal cell density for obtaining the highest virus production may vary according to the cell type used for the virus propagation.

The inoculation can be carried out at an MOI (Multiplicity Of Infection) of about 10⁻¹ to 10⁻⁷, suitably about 10⁻² to 10⁻⁶, and more suitably, about 10⁻⁵.

The temperature and pH conditions for virus infection may vary. Temperature may range from 32° C. to 39° C. depending on the virus type. For Influenza virus production, cell culture infection may vary depending on the strain which is produced. Influenza virus infection is suitably performed at a temperature ranging from 32° C. to 35° C., suitably at 33° C. In one embodiment, the virus infection occurs at 33° C. In an alternative embodiment, the virus infection takes place at 35° C. Proteases, typically trypsin, may be added to the cell culture depending on the virus strain, to allow viral replication. The protease can be added at any suitable stage during the culture. Tryspin is preferably of non-animal origin, that is to say the protease is not purified from an animal source. It is suitably recombinantly produced in a micro-organism, such as bacterial, yeast or plant. Suitable examples of recombinant trypsin are Trypzean, a recombinant trypsin produced in corn (Prodigen, 101 Gateway Blvd, Suite 100 College Station, Texas 77845. Manufacturer code: TRY), or TrpLE (Invitrogen) which is a trypsin-like enzyme expressed in fungus (WO2004/020612).

Once infected, cells may release into the culture medium newly formed virus particles, due to spontaneous lysis of host cells, also called passive lysis. Therefore, in one embodiment, cell-produced viral harvest may be provided any time after virus inoculation by collecting the cell culture medium. This mode of harvesting is particularly suitable when it is desired to harvest cell-produced virus at different time points after virus inoculation, and pooling the different harvests, if needed.

Alternatively, after virus infection, cell culture-based virus may be harvested by employing external factor to lyse host cells, also called active lysis. However, contrary to the previous one, such a harvesting mode requires that the cell-derived viral harvest be collected at a single time point, as actively lysing the cells will immediately terminate the cell culture.

Methods that can be used for active cell lysis are known to the person skilled in the art. Useful methods in this respect are for example, freeze-thaw, solid shear, hypertonic and/or hypotonic lysis, liquid shear, high-pressure extrusion, detergent lysis, or any combination thereof.

According to one embodiment, cell culture-based viral harvest may be provided any time after virus inoculation by collecting the cell culture supernatants, lysing the inoculated cells or both.

Before harvesting, cell infection may last for 2 to 10 days. According to a specific embodiment, culture supernatants from days 3, 4 and 5 post-inoculation are harvested and pooled for further downstream processing (virus isolation). According to a distinct embodiment, cell culture supernatant is collected from day 5 post-inoculation. The optimal time to harvest the cell-produced virus is usually based on the determination of the infection peak. For example, the CPE (CytoPathic Effect) is measured by monitoring the morphological changes occurring in host cells after virus inoculation, including cell rounding, disorientation, swelling or shrinking, death, detachment from the surface. The detection of a specific viral antigen may also be monitored by standard techniques of protein detection, such as a Western-blot analysis. Harvest can then be collected when the desired detection level is achieved. In the particular case of influenza virus, the content of HA may be monitored any time post-inoculation of the cells with the virus, by the SRD assay (Wood, J M, et al. (1977). J. Biol. Standard. 5, 237-247), which is a technique familiar to a person skilled in the art. Additionally, the SRD assay may also be used for determining the optimal cell density range required to obtain an optimized virus yield.

In the context of the present invention, the cell culture phase is to be understood as encompassing any step preceding the virus collecting step, while the virus purification phase is to be understood as encompassing any step following said collecting step. According to the present invention, the virus purification phase includes at least one homogenization step, implemented at any appropriate time of the process. Accordingly, in a specific embodiment of the invention, the virus purification phase comprises, in addition to at least one homogenization step, at least one step selected from clarification, nucleic acid degradation, ultrafiltration/diafiltration, density gradient ultracentrifugation and chromatography, or any combination thereof. Depending on the purity level that is desired, the above steps may be combined in any way. In a specific embodiment, at least one HPH step is implemented in combination with any of the above purification step, when said above purification step is implemented during the virus purification phase. The present invention contemplates, for example, that HPH be implemented before clarification, or before nucleic acid degradation, or before sucrose gradient ultracentrifugation, when the indicated step is implemented. The invention also provides for a combination of purification steps. Therefore according to a specific embodiment, homogenization, in particular HPH, is implemented after the successive steps of clarification, ultrafiltration and, optionally, Benzonase™. This combination, clarification/ultrafiltration/Benzonase™/HPH may also be associated with a further purification step, for instance, a sucrose gradient ultracentrifigation step and this latter combination forms another object of the present invention.

According to the method of the invention, it may be possible to combine a purification step, such as sucrose gradient ultracentrifugation, with a virus splitting step. In particular, a splitting agent may be added to the sucrose gradient. This embodiment is particularly suitable, when it is desired to minimize the total number of steps of the method of the invention, as it allows, within a single operation, to both purify and split the virus. Hence, in certain embodiments, when at least one sucrose gradient ultracentrifugation is implemented, the sucrose gradient additionally comprises a splitting agent.

Alternatively, the virus splitting step of the method of the present invention, when implemented, is performed in batch.

At the end of the virus purification phase, the virus preparation obtained according to the method of the present invention may be suitably subjected to sterile filtration, as is common in processes for pharmaceutical grade materials, such as immunogenic compositions or vaccines, and known to the person skilled in the art. Such sterile filtration can for instance suitably be performed by filtering the preparation through a 0.22 μm filter. After sterile preparation, the virus or viral antigens are ready for clinical use, if desired.

The immunogenic compositions, in particular vaccines, may generally be formulated in a sub-virion form, e.g. in the form of a split virus, where the lipid envelope has been dissolved or disrupted, or in the form of one or more purified viral proteins (subunit vaccine). As an alternative, the immunogenic compositions may include a whole virus, e.g. a live attenuated whole virus, or an inactivated whole virus.

Methods of splitting viruses, such as influenza viruses, are well known in the art (WO02/28422). Splitting of the virus is carried out by disrupting or fragmenting whole virus whether infectious (wild-type or attenuated) or non-infectious (inactivated) with a disrupting concentration of a splitting agent. Splitting agents generally include agents capable of breaking up and dissolving lipid membranes. Traditionally, split influenza virus was produced using a solvent/detergent treatment, such as tri-n-butyl phosphate, or diethylether in combination with Tween™ (known as “Tween-ether” splitting) and this process is still used in some production facilities. Other splitting agents now employed include detergents or proteolytic enzymes or bile salts, for example sodium deoxycholate. Detergents that can be used as splitting agents include cationic detergents e.g. cetyl thrimethyl ammonium bromide (CTAB), other ionic detergents, e.g. sodium lauryl sulphate (SLS), taurodeoxycholate, or non-ionic detergents such as Tween or Triton X-100, or combination of any two or more detergents.

In one embodiment, the splitting agent is deoxycholate. In another embodiment, the splitting agent is Triton X-100. In a further embodiment, the method according to the invention uses a combination of Triton X-100 and sodium lauryl sulfate as splitting agents.

The splitting process may be carried out as a batch, continuous or semi-continuous process. When implemented in batch, the split virus may require an additional step of purification, such as a chromatography step. It is not necessary to implement a splitting step as such, as it is possible to perform the splitting simultaneously to a purification step. For instance, a detergent may be added to the sucrose gradient aimed at purifying viral proteins by ultracentrifugation, as described above. In one embodiment, the method according to the invention comprises a splitting step performed in batch with a detergent, in particular, Triton X-100, in addition to at least one homogenization step.

For the safety of vaccines, it may be necessary to reduce infectivity of the virus suspension along different steps of the purification process. The infectivity of a virus is determined by its capacity to replicate on a cell line. Therefore, the method according to the present invention, optionally, includes at least one virus inactivation step. The inactivation may be performed by using, for instance, beta-propiolactone (BPL), formaldehyde, or UV, or any combination thereof, at any suitable step of the method. In one specific embodiment, the method according to the invention further comprises at least one BPL treatment step. In a specific embodiment, the method according to the invention further comprises at least one BPL treatment step and at least one formaldehyde treatment step. Formaldehyde and BPL may be used sequentially, in any order, for instance, formaldehyde is used after the BPL. In one embodiment, the formaldehyde treatment is followed by at least one homogenization step. When using, in particular, UV as the inactivation method, implementing homogenization of the virus preparation before UV irradiation, may help improve the efficiency of the virus inactivation. Viruses, or part of the viruses, which would be present within aggregates, whether virus aggregates or virus/cell aggregates may escape to irradiation because of burial within said aggregates, and, thus, non-accessibility of some virus or virus part to the inactivation agent. In one embodiment, the virus preparation obtained according to the method of the present invention is inactivated, for instance, by UV irradiation, and homogenization is performed immediately before said inactivation. Accordingly, in a specific embodiment of the present invention, the virus-containing cell culture medium is clarified and homogenized by HPH and further treated with UV. In an alternative embodiment, the virus-containing cell culture medium is successively homogenized by HPH, clarified by filtration or centrifugation and further treated with BPL or UV. The conditions of viral inactivation may vary and will be determined, in particular, by assessing the residual virus infectivity by measuring the Tissue Culture Infectious dose (TCID₅₀/ml).

Immunogenic compositions of the present invention, including vaccines, can optionally contain the additives customary for vaccines, in particular substances which increase the immune response elicited in a patient who receives the composition, i.e. so-called adjuvants.

In one embodiment, immunogenic compositions are contemplated, which comprise a virus or viral antigen of the present invention admixed with a suitable pharmaceutical carrier. In a specific embodiment, they comprise an adjuvant.

Adjuvant compositions may comprise an oil in water emulsion which comprise a metabolisable oil and an emulsifying agent. In order for any oil in water composition to be suitable for human administration, the oil phase of the emulsion system has to comprise a metabolisable oil. The meaning of the term metabolisable oil is well known in the art. Metabolisable can be defined as ‘being capable of being transformed by metabolism’ (Dorland's Illustrated Medical Dictionary, W.B. Sanders Company, 25th edition (1974)). The oil may be any vegetable oil, fish oil, animal oil or synthetic oil, which is not toxic to the recipient and is capable of being transformed by metabolism. Nuts, seeds, and grains are common sources of vegetable oils. Synthetic oils are also part of this invention and can include commercially available oils such as NEOBEE® and others.

A particularly suitable metabolisable oil is squalene. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is a particularly preferred oil for use in this invention. Squalene is a metabolisable oil by virtue of the fact that it is an intermediate in the biosynthesis of cholesterol (Merck index, 10th Edition, entry no. 8619). In a further embodiment of the invention, the metabolisable oil is present in the immunogenic composition in an amount of 0.5% to 10% (v/v) of the total volume of the composition.

The oil-in-water emulsion further comprises an emulsifying agent. The emulsifying agent may suitably be polyoxyethylene sorbitan monooleate. Further, said emulsifying agent is suitably present in the vaccine or immunogenic composition 0.125 to 4% (v/v) of the total volume of the composition.

The oil-in-water emulsion of the present invention optionally comprises a tocol. Tocols are well known in the art and are described in EP0382271. Suitably may be a tocol is alpha-tocopherol or a derivative thereof such as alpha-tocopherol succinate (also known as vitamin E succinate). Said tocol is suitably present in the adjuvant composition in an amount 0.25% to 10% (v/v) of the total volume of the immunogenic composition.

The method of producing oil-in-water emulsions is well known to the person skilled in the art. Commonly, the method comprises mixing the oil phase (optionally comprising a tocol) with a surfactant such as a PBS/TWEEN80™ solution, followed by homogenisation using a homogenizer, it would be clear to a man skilled in the art that a method comprising passing the mixture twice through a syringe needle would be suitable for homogenising small volumes of liquid. Equally, the emulsification process in microfluidiser (M110S Microfluidics machine, maximum of 50 passes, for a period of 2 minutes at maximum pressure input of 6 bar (output pressure of about 850 bar)) could be adapted by the man skilled in the art to produce smaller or larger volumes of emulsion. The adaptation could be achieved by routine experimentation comprising the measurement of the resultant emulsion until a preparation was achieved with oil droplets of the required diameter.

In an oil-in-water emulsion, the oil and emulsifier are in an aqueous carrier. The aqueous carrier may be, for example, phosphate buffered saline.

In particular, the oil-in-water emulsion systems of the present invention have a small oil droplet size in the sub-micron range. Suitably the droplet sizes will be in the range 120 to 750 nm, more particularly sizes from 120 to 600 nm in diameter. Even more particularly, the oil-in water emulsion contains oil droplets of which at least 70% by intensity are less than 500 nm in diameter, more particular at least 80% by intensity are less than 300 nm in diameter, more particular at least 90% by intensity are in the range of 120 to 200 nm in diameter.

The oil droplet size, i.e. diameter, according to the present invention is given by intensity. There are several ways of determining the diameter of the oil droplet size by intensity. Intensity is measured by use of a sizing instrument, suitably by dynamic light scattering such as the Malvern Zetasizer 4000 or suitably the Malvern Zetasizer 3000HS. A detailed procedure is given in Example II.2. A first possibility is to determine the z average diameter ZAD by dynamic light scattering (PCS-Photon correlation spectroscopy); this method additionally give the polydispersity index (PDI), and both the ZAD and PDI are calculated with the cumulants algorithm. These values do not require the knowledge of the particle refractive index. A second mean is to calculate the diameter of the oil droplet by determining the whole particle size distribution by another algorithm, either the Contin, or NNLS, or the automatic “Malvern” one (the default algorithm provided for by the sizing instrument). Most of the time, as the particle refractive index of a complex composition is unknown, only the intensity distribution is taken into consideration, and if necessary the intensity mean originating from this distribution.

The adjuvant compositions may further comprise a Toll like receptor (TLR) 4 agonist. By “TLR4 agonist” it is meant a component which is capable of causing a signalling response through a TLR4 signalling pathway, either as a direct ligand or indirectly through generation of endogenous or exogenous ligand (Sabroe et al, JI 2003 p1630-5). The TLR 4 may be a lipid A derivative, particularly monophosphoryl lipid A or more particularly 3 Deacylated monophoshoryl lipid A (3 D-MPL).

3D-MPL is available under the trademark MPL® by GlaxoSmithKline Biologicals North America and primarily promotes CD4+ T cell responses with an IFN-g (Th1) phenotype. It can be produced according to the methods disclosed in GB 2 220 211 A. Chemically it is a mixture of 3-deacylated monophosphoryl lipid A with 3, 4, 5 or 6 acylated chains. In particular, in the adjuvant compositions of the present invention small particle 3 D-MPL is used. Small particle 3 D-MPL has a particle size such that it may be sterile-filtered through a 0.22 μm filter. Such preparations are described in International Patent Application No. WO 94/21292. Synthetic derivatives of lipid A are known and thought to be TLR 4 agonists including, but not limited to:
OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026)
OM 294 DP (3S,9R)-3-[(R)-dodecanoyloxytetradecanoylamino]-4-oxo-5-aza-9(R)-[(R)-3-hydroxytetradecanoylamino]decan-1,10-diol,1,10-bis(dihydrogenophosphate) (WO99/64301 and WO 00/0462)
OM 197 MP—Ac DP (3S—,9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127)

Other TLR4 ligands which may be used are alkyl Glucosaminide phosphates (AGPs) such as those disclosed in WO9850399 or U.S. Pat. No. 6,303,347 (processes for preparation of AGPs are also disclosed), or pharmaceutically acceptable salts of AGPs as disclosed in U.S. Pat. No. 6,764,840. Some AGPs are TLR4 agonists, and some are TLR4 antagonists. Both are thought to be useful as adjuvants. In addition, further suitable TLR-4 agonists are disclosed in US2003/0153532 and US2205/0164988.

The invention is particularly suitable for preparing influenza virus immunogenic compositions, including vaccines. Various forms of influenza virus are currently available. They are generally based either on live virus or inactivated virus. Inactivated vaccines may be based on whole virions, spilt virions or purified surface antigens (including HA). Influenza antigens can also be presented in the form of virosomes (nucleic acid-free viral-like liposomal particles).

Virus inactivation methods and splitting methods have been described above and are applicable to influenza virus.

Influenza virus strains for use in vaccines change from season to season. In the current inter-pandemic period, vaccines typically include two influenza A strains and one influenza B strain. Trivalent vaccines are typical, but higher valence, such as quadrivalence, is also contemplated in the present invention. The invention may also use HA from pandemic strains (i.e. strains to which the vaccine recipient and the general human population are immunologically naïve), and influenza vaccines for pandemic strains may be monovalent or may be based on a normal trivalent vaccine supplemented by a pandemic strain.

Compositions of the invention may include antigen(s) from one or more influenza virus strains, including influenza A virus and/or influenza B virus. In particular, a trivalent vaccine including antigens from two influenza A virus strains and one influenza B virus strain is contemplated by the present invention. Alternatively a quadrivalent vaccine including antigens from two influenza A virus strains and two influenza B virus strains is also within the scope of the present invention.

The compositions of the invention are not restricted to monovalent compositions, i.e. including only one strain type, i.e. only seasonal strains or only pandemic strains. The invention also encompasses multivalent compositions comprising a combination of seasonal strains and/or of pandemic strains. In particular, a quadrivalent composition, which may be adjuvanted, comprising three seasonal strains and one pandemic strain falls within the scope of the invention. Other compositions falling within the scope of the invention are a trivalent composition comprising two A strains and one B strain, such as H1N1, H3N2 and B strains, and a quadrivalent composition comprising two A strains and two B strains of a different lineage, such as H1N1, H3N2, B/Victoria and B/Yamagata.

HA is the main immunogen in current inactivated influenza vaccines, and vaccine doses are standardized by reference to HA levels, typically measured by SRD. Existing vaccines typically contain about 15 μg of HA per strain, although lower doses can be used, e.g. for children, or in pandemic situations, or when using an adjuvant. Fractional doses such as a half (i.e. 7.5 μg HA per strain) or a quarter have been used, as have higher doses, in particular, 3× or 9× doses. Thus immunogenic compositions of the present invention may include between 0.1 and 150 μg of HA per influenza strain, particularly, between 0.1 and 50 μg, e.g. 0.1-20 μg, 0.1-15 μg, 0.1-10 μg, 0.1-7.5 μg, 0.5-5 μg, etc. Particular doses include about 15, about 10, about 7.5, about 5 μg per strain, about 3.8 μg per strain and about 1.9 μg per strain.

Once an influenza virus has been purified for a particular strain, it may be combined with viruses from other strains to make a trivalent vaccine, for example, as described above. It is more suitable to treat each strain separately and to mix monovalent bulks to give a final multivalent mixture, rather than to mix viruses and degrade DNA and purify it from a multivalent mixture.

The invention will be further described by reference to the following, non-limiting, examples.

In the following examples, influenza virus is produced by cell culture and is harvested by collecting the cell culture supernatant after infection of cells with the virus and its release into the cell culture medium. Therefore, in all examples below, the expressions “virus harvest” or “viral harvest” both refer to the influenza virus-containing cell culture supernatant or cell culture mediul.

In the following examples, pressure is indicated either in bars or in psi (pound per square inch). Bars and psi can be converted into one another by using the following conversion formula: 1 bar=14.5 psi

### Example I

### Homogenization of Influenza Viral Harvest Prior to Clarification Results in a Higher Virus Yield after Said Clarification

### I.1. MDCK Cell Culture Conditions and Virus Infection (NCP124, NCP127, NCP134, NYP132, NYP133, JP115, JP128, JP129, JP130, JP131, EFC3APA001 and EFC3APA002)

MDCK adherent cells were grown on microcarriers in perfusion culture mode at 36.5° C. After the growth phase, once the appropriate cell density was reached (above 5×10⁶ cells/ml), cells were inoculated with Influenza virus (Multiplicity of Infection of 1×10⁻⁵) in perfusion mode and the temperature was switched to 33° C. The virus was harvested by perfusion at days 3, 4 and 5 post-inoculation. The perfusion harvests were pooled and stored at a temperature ranging from 2 to 4° C. until further processing.

After harvesting the influenza virus-containing cell culture medium, an immediate clarification of the viral harvest is typically responsible for the loss of around 30% of the virus. This assertion is illustrated in Table 1 presenting the influenza virus yields obtained in numerous independent experiments, whether of A strain or B strain, which were obtained after clarification on a filtration train (Pall) composed of three different depth filters with nominal porosities of Profile 5 μm (Ref: BYA050P6)—Profile 0.5 μm (Ref: BYY005P6)—Preflow 0.2 μm (Ref: DFA3001UUAC), sharing all the absence of a prior homogenization step. The influenza virus yield was evaluated by measuring the HA content before and after clarification according to the SRD assay, as described below in section I.6. Results are presented in Table 1 in the form of percentages to be compared to the control value 100% representing the total HA amount present in the starting material, i.e. present in the viral harvest before clarification.

TABLE 1
HA yield after clarification-Influenza virus produced on MDCK cells

| | |
|---|---|
| | Clarification |
| Experiment | HA yield (%) |

A/New Caledonia strain

| | |
|---|---|
| NCP124 | 93 |
| NCP127 | 71 |
| NCP134 | 62 |

A/New York strain

| | |
|---|---|
| NYP132 | 54 |
| NYP133 | 55 |

B/Jiangsu strain

| | |
|---|---|
| JP125 | 90 |
| JP128 | 65 |
| JP129 | 72 |
| JP130 | 47 |
| JP131 | 78 |
| EFC3APA001 | 64 |
| EFC3APA002 | 59 |
| Average HA yield | 67.5 |

Results—Conclusions:

The average yield obtained after clarification is around 70%, indicating that around 30% of virus amount are lost during the clarification step.

### I.2. MDCK Cell Culture Conditions and Virus Infection—WiP136, WiP144, WiP145, SOP138, SOP151, MAP140 and MAP142

Cell culture and virus infection were performed as described in section I.1., except that in the experiment WiP144, the virus was used at a Multiplicity of Infection of 1×10⁻⁶. The virus was harvested by perfusion at days 3, 4 and 5 post-inoculation. The perfusion harvests were pooled and stored at a temperature ranging from 2 to 4° C. until further processing.

Contrary to what was observed in Table 1, when implementing a prior homogenization step, whether high-pressure homogenization or sonication, the virus loss obtained after clarification is strongly reduced and only barely noticeable. This is illustrated in Table 2 presenting numerous HA yields, whether of A strain or B strain (as measured by SRD assay as described below in section I.6), which were obtained after clarification. In every experiment, a homogenization step (whether HPH or sonication) was implemented prior to clarification, as indicated in Table 2. In the experiments WiP144, SOP138, SOP151, MaP140, Map142 and WiP136, clarification was performed on a filtration train (Pall) composed of three different depth filters with nominal porosities of Profile 5 μm (Ref: BYA050P6)—Profile 0.5 μm (Ref: BYY005P6)—Preflow 0.2 μm (Ref: DFA3001UUAC). In the experiment WiP145, the collected virus-containing culture medium was first pre-clarified on a Profile 1 μm filter, then homogenized by HPH (at 2 different pressures, 700 bars and 1000 bars), and further clarified on a Preflow 0.2 μm filter. Results are presented in Table 2 in the form of percentages to be compared to the control value 100% representing the total HA amount present in the starting material, i.e. present in the untreated viral harvest.

TABLE 2
Effect of a prior homogenization step on HA yield after
clarification-Influenza virus produced on MDCK cells

| | Homogenization | | |
|---|---|---|---|
| | HPH* | Sonication | Clarification |
| Experiment | (pressure in bars) | (amplitude %) | HA yield (%) |

A/Wisconsin strain

| | | | |
|---|---|---|---|
| WiP136 | 700 | — | 101 |
| | — | 70 | 116 |
| WiP144 | 700 | — | 70** |
| WiP145 | 700 | — | 91 |
| | 1000 | | 94 |

A/Solomon Island strain

| | | | |
|---|---|---|---|
| SOP138 | 700 | — | 93 |
| SOP151 | 700 | — | 86 |

B/Malaysia strain

| | | | |
|---|---|---|---|
| MaP140 | 700 | — | 102 |
| | — | 80 | 96 |
| MaP142 | 700 | — | 95 |
| Average HA | | | 97 |
| yield | | | |

*High-pressure Homogenization,
**plugging

Results—Conclusions

The average yield obtained after clarification was around 100%, indicating that only minor loss, if any, occurred during the clarification step when a prior homogenization step has been performed. It should be noted that (i) both A and B strains provided clarification yields close to 100% and (ii) such a high yield was obtained with both high-pressure homogenization and sonication.

Interestingly, during the experiment (WiP144) displaying the only low clarification yield of 70%, a plugging of the filter occurred during the clarification, due to the filter which appeared to be undersized. Homogenization is supposed to prevent or limit filter plugging by decreasing the amount and size of aggregates, providing thus a higher yield. Therefore, the low yield obtained with WiP144 associated with a filter plugging reinforces that hypothesis. For that reason, WiP144 was excluded from the average calculation.

### I.3. EB66® Cell Culture Conditions and Virus Infection—EB66 17

EB66® cells were grown in suspension in a batch mode. They were infected with H₅N₁ and the virus was harvested by collecting the cell culture medium a few days later. After harvesting the influenza virus-containing cell culture medium, an immediate clarification of the viral harvest by centrifugation at 300 g for 15 minutes is responsible for the loss of around 50% of the virus. As observed for influenza virus produced on MDCK cells in Table 2, implementing a homogenization step prior to clarification by centrifugation results in a strong decrease of the virus loss after clarification, as analysed by measuring the HA content before and after clarification. Results are presented in Table 3 in the form of percentages to be compared to the control value 100% representing the total HA amount present in the starting material, i.e. present in the untreated viral harvest.

TABLE 3
Effect of a prior homogenization step on HA yield after clarification -
Influenza virus produced on EB66 ® cells

| | | Homogenization | | | | | |
|---|---|---|---|---|---|---|---|
| | No | Sonication | | | HPH* | | |
| | Homoge- | Amplitude (%) | | | Pressure in bars | | |
| EB66_17 | nization | 40% | 60% | 80% | 100 | 150 | 200 |
| Clarification | 43.1 | 86.5 | 87.3 | 83.2 | 81.3 | 96.2 | 87.1 |
| HA yield (%) | | | | | | | |

*High-pressure Homogenization

### I.4. High-Pressure Homogenization

The homogenizer used in this study was the Rannie™ device MiniLab 7.30VH (from APV) consisting of 2 pistons which work in alternation. A peristaltic pump is used to maintain a constant flow through the homogenizer. The pump forces the product under pressure through a small adjustable gap. The device is used according to the manufacturer's recommendations. Virus-containing cell culture medium collected from the indicated experiments were homogenized at a flow rate of around 100 ml/min and at a pressure of 700 bars or 1000 bars, as indicated in Table 2, or 100. 150 and 200, as indicated in Table 3. The homogenized fluid was recovered on ice to limit the temperature increase. In some experiments, high-pressure homogenization was performed using the Panda™ device from Niro Soavi (SOP151).

### I.5. Sonication

Sonication was performed with the Sonics Vibra-CeII™ CV33 sonicator from Sonics & Materials. Virus-containing cell culture medium collected from the indicated experiments were sonicated at a flow rate of 70 ml/min and at an amplitude of 80%, 60%, 70% or 40%, as indicated in Tables 2 and 3. The sonicated product was collected on ice to limit the temperature increase.

### I.6. SRD Method Used to Measure HA Content

Glass plates (12.4-10 cm) were coated with an agarose gel containing a concentration of anti-influenza HA serum that is recommended by NIBSC. After the gel has set, 72 sample wells (3 mm diameter) were punched into the agarose. 10 μl of appropriate dilutions of the reference and the sample were loaded in the wells. The plates were incubated for 24 hours at room temperature (20 to 25° C.) in a moist chamber. After that, the plates were soaked overnight with NaCl solution and washed briefly in distilled water. The gel was then pressed and dried. When completely dry, the plates were stained on Coomassie Brillant Blue solution for 10 minutes and destained twice in a mixture of methanol and acetic acid until clearly defined stained zones become visible. After drying the plates, the diameter of the stained zones surrounding antigen wells was measured in two directions at right angles. Alternatively equipment to measure the surface can be used. Dose-response curves of antigen dilutions against the surface were constructed and the results were calculated according to standard slope-ratio assay methods (Finney, D. J. (1952). Statistical Methods in Biological Assay. London: Griffin, Quoted in: Wood, J M, et al (1977). J. Biol. Standard. 5, 237-247).

### Example II

### Homogenization Effect on Clarification Yield: Comparison Between High-Pressure Homogenization and Sonication

Two homogenization techniques, high-pressure homogenization (HPH) (700 bars) and sonication (amplitude 80%), were compared by analysing the respective effect of implementing one of them on the virus-containing cell culture medium prior to clarification. For each technique, clarifications on Profile (5 μm/0.5 μm) and Preflow (0.2 μm) filters were also tested in parallel. HPH and sonication were conducted as described in Example I, sections I.4. and I.5., respectively.

HA yields after homogenization and after clarification were calculated according to the SRD assay, as described in Example I (I.6. section). Results are presented in Table 4 in the form of percentages to be compared to the control value 100% representing the total HA amount present in the starting material, i.e. present in the untreated viral harvest.

TABLE 4
Effect of a prior HPH step or sonication
step on HA yield after clarification

| | | | Clarification - Type of filter - | | | |
|---|---|---|---|---|---|---|
| | Homogenization | | Profile 5/0.5 μm | | Preflow 0.2 μm | |
| B/Malaysia | | Soni- | | soni- | | soni- |
| strain | HPH* | cation | HPH* | cation | HPH* | cation |
| HA yield (%) | 113 | 93 | 102 | 100 | 102 | 96 |

*High-pressure Homogenization

Results—Conclusions

None of the homogenization technique (HPH or sonication) significantly affect, per se, the HA content (Homogenization columns). As observed in Table 2, when a prior homogenization step is implemented, whether HPH or sonication, HA clarification yields reach around 100%, irrespective of the type of filter used for clarification (Clarification columns). There is no significant difference, in terms of HA clarification yields between HPH and sonication, indicating that both homogenization techniques help limit the virus loss occurring during the clarification step.

### Example III

### Direct Effect of Prior Homogenization on Clarification Yield by Comparing the Virus Yield Obtained after Clarification without a Prior Homogenization and the Virus Yield Obtained after Homogenization and Clarification

To demonstrate the positive impact of homogenization on clarification, a series of four experiments was carried out in duplicate. Each one of these experiments was performed according to specific clarifications conditions. Within each experiment, one duplicate was subjected to homogenization before clarification, whereas the second one was clarified, according to the same conditions as the other duplicate, with no prior homogenization, providing thus an internal control allowing to assess the direct effect of homogenization on the yield obtained after each clarification condition.

Cell culture and virus infection were performed as described in sections I.1. and I.2. of Example I.

The perfusion harvests were pooled and separated into two parts. One part was treated as the control experiment, where no prior homogenization was performed before clarification and the other part was homogenized by high-pressure homogenization before clarification. 2 series of experiments were run: (i) homogenization was performed directly on the virus harvest (WiP136), or (ii) the virus harvest was first pre-clarified and then homogenized (WiP137, WiP137bis and MaP141). In any case, the homogenized harvest, whether or not a prior pre-clarification was carried out, was subjected to a clarification step.

### III.1. Experiment WiP136

### III.1.1 Control Experiment (No Prior Homogenization):

A volume of 2 liters of virus harvest was filtrated onto a Millistack COHC filter (Millipore, cat No. MC0HC23HH3) of a 23 cm² surface which was previously conditioned with PBS-citrate 125 mM-NaCl 1M, pH 7.4. The maximum inlet pressure of this filter is 2.1 bars. The loading flow rate was 20 ml/min. The pressure was recorded all along the filtration and the experiment was stopped when the pressure reached 1.5 bars. The filter was washed with PBS-Citrate 125 mM-NaCl 1 M, pH 7.4 and the wash volume was pooled with the filtrated harvest. The total volume recovered was 695 ml. HA content was measured on the total volume.

### III.1.2. Homogenization Experiment:

A volume of 2 liters of virus harvest was homogenized with the Rannie™ device MiniLab 7.30VH system (APV) at a flow rate of 110 ml/min and at a pressure of 700 bars. The homogenized harvest was recovered on ice to limit the temperature increase. The Rannie™ device was washed with PBS-Citrate 125 mM, pH 7.4 and the wash buffer was pooled with the homogenized harvest to give a final volume of 2.2 liters. The homogenized harvest was loaded onto a Millistack COCHC filter which was previously conditioned with PBS-Citrate 125 mM-NaCl 1M, pH 7.4. The loading flow rate was 20 ml/min. The pressure was recorded all along the filtration and the experiment was stopped when the pressure reached 1.6 bars. A volume of 1350 ml was filtrated. After loading, the filter was washed with PBS-Citrate 125 mM-NaCl 1M, pH 7.4 and the wash volume was pooled with the filtrated harvest to give a clarified harvest volume of 1460 ml. HA content was measured on the total volume.

### III.2. Experiment WiP137

In this experiment, the harvest was pre-clarified and concentrated prior to be homogenized. A volume of 2 liters of virus harvest was filtrated onto a Mini Profile II 5 μm (Pall) filter of 90 cm². A pre-clarified harvest of 4649 g was recovered. The pre-clarified harvest was then concentrated 12 times on three Millipore membranes with cut-off of 300 kD, 500 kD and 100 kD. The three retentates were pooled to give a volume of 300 ml. Said volume was split into 2 fractions of 150 ml.

### III.2.1. Control Experiment (No Prior Homogenization):

A volume of 150 ml of retentate was loaded onto a Millistak COHC filter (Millipore, cat No. MCOHC23HH3) of a 23 cm² surface which was previously conditioned with PBS-Citrate 125 mM-NaCl 1 M, pH 7.4. The average loading flow rate was 16 ml/min. The pressure was recorded all along the filtration and the experiment was stopped when the totality of the sample was filtrated. The total weight of the filtrate was 230 g. HA content was measured on the filtrate.

### III.2.2. Homogenization Experiment:

A volume of 150 ml of retentate was homogenized with the Rannie™ device MiniLab 7.30VH at a flow rate of 100 ml/min and at a pressure of 700 bars. The homogenized harvest was recovered on ice to limit the temperature increase. The Rannie™ device was washed with PBS-Citrate 125 mM, pH 7.4 and the wash buffer was pooled with the homogenized harvest to give a final volume of 232 ml. The homogenized retentate was loaded onto a Millistak COHC filter which was previoulsy conditioned with PBS-Citrate 125 mM-NaCl 1 M, pH 7.4. The average loading flow rate was 19 ml/min. The pressure was recorded all along the filtration and the experiment was stopped when the totality of the sample was filtrated. HA content was measured on the filtrate.

### III.3. Experiment WiP137bis

In this experiment, the harvest was pre-clarified prior to be homogenized. A volume of 20 liters of virus harvest was filtrated on two Profile 5 μm filters (Pall) of 90 cm² at an average flow rate of 54 ml/min. The clarified harvest was then filtrated onto a Profile 5 μm (Pall) of 450 cm² at a flow rate of 476 ml/min. The filter was washed with PBS-Citrate 125 mM, pH 7.4 to give an overall pre-clarified harvest weight of 27617 g. A volume of 21.5 liters of pre-clarified harvest was then concentrated 11 times on a Ultracell 100 kD cut-off membrane (Millipore) of 0.1 m².

### III.3.1. Control Experiment (No Prior Homogenization):

A volume corresponding to 500 g of the above Ultracell 100 kD retentate was loaded onto a Millistak COHC filter (Millipore, cat. No. MCOHC23HH3) of a 23 cm² surface which was previously conditioned with PBS-Citrate 125 mM-NaCl 1 M, pH 7.4. The maximum inlet pressure of this filter is 1.4 bars. The average loading flow rate was 13 ml/min. The pressure was recorded all along the filtration and the experiment was stopped when the pressure reached 20 psi. The filter was successively washed with PBS-Citrate 125 mM, pH 7.4, PBS-Citrate 125 mM, NaCl 1 M, pH 7.4 and the wash volumes were pooled with the filtrated harvest. The total volume of the filtrate was 287 ml. HA content was measured on the total volume.

### III.3.2. Homogenization Experiment:

A volume corresponding to 930 g of the above Ultracell 100 kD retentate was homogenized with the Rannie™ device at a flow rate of 110 ml/min and at a pressure of 700 bars. The homogenized harvest was recovered on ice to the temperature increase. The Rannie™ device was washed with PBS-Citrate 125 mM, pH 7.4 and the wash buffer was pooled with the homogenized harvest to give a final volume of 1035 ml. The volume corresponding to 500 g of homogenized retentate was loaded onto a Millistak COHC filter which was previously conditioned with PBS-Citrate 125 mM-NaCl 1 M, pH 7.4. The average loading flow rate was 14 ml/min. The pressure was recorded all along the filtration and the experiment was stopped when the totality of the sample was filtrated. After loading, the filter was washed with PBS-Citrate 125 mM, pH 7.4 and the wash volume was pooled with the filtrated harvest to give a final clarified and homogenized harvest volume of 670 ml. HA content was measured on the final volume.

### III.4. Experiment MaP141

In this experiment, the harvest was pre-clarified prior to be homogenized. A volume of 8 liters of virus harvest was filtrated onto a Profile 1 μm filter (Pall) of 90 cm² at an average flow rate of 18.5 ml/min. The filter was washed with PBS-Citrate 125 mM, pH 7.4 to give an overall pre-clarified harvest weight of 8328 g. A volume of 6.5 liters of pre-clarified harvest was concentrated 5 times on a Ultracell 1000 kD cut-off membrane (Millipore) of 0.1 m² and a volume of 1.5 liter of pre-clarified harvest was concentrated on a Ultracell 1000 kD cut-off membrane (Millipore) of 0.005 m². The two Ultracell retentates were pooled.

### III.4.1. Control Experiment (No Prior Homogenization)

A volume of 160 ml of the above pooled retentates was loaded onto a Preflow 0.2 μm filter of 13.6 cm² which was previously conditioned with PBS-Citrate 125 mM-NaCl 0.3M, pH 7.4. The average loading flow rate was 4.3 ml/min. The pressure was recorded all along the filtration and the experiment was stopped when the pressure reached 20 psi. The filter was washed with PBS-Citrate 125 mM-NaCl 1 M, ph 7.4 and the wash volume was pooled with the filtrated harvest. The total volume of the filtrate was 87 ml. HA content was measured on the total volume.

### III.4.2. Homogenization Experiment

A volume of 1120 ml of the above retentate was homogenized with the Rannie™ device at a flow rate of 110 ml/min and at a pressure of 700 bars. The homogenized harvest was recovered on ice to limit the temperature increase. The Rannie™ device was washed with PBS-Citrate 125 mM, pH 7.4 and the wash volume was pooled with the homogenized harvest to give a final volume of 1341 ml. A volume of 192 ml of homogenized retentate was loaded onto a Preflow 0.1 μm filter of 13.6 cm² which was previously conditioned with PBS-Citrate 125 mM-NaCl 0.3 M, pH 7.4. The average loading flow rate was 4.7 ml/min. The pressure was recorded all along the filtration and the experiment was stopped when the totality of the sample was filtrated. After loading, the filter was washed with PBS-Citrate 125 mM-NaCl 1 M pH 7.4 and the wash volume was pooled with the filtrated harvest to give a clarified harvest volume of 253 ml. HA content was measured on the total volume.

The impact of performing a homogenization step before clarification was assessed by evaluating the HA yield obtained after clarification, in the presence or absence of a prior homogenization step. HA content is measured by the SRD assay, as described in Example I (section I.6.). In both control experiments (with no prior homogenization) and homogenization experiments, HA was first measured on the untreated viral harvest and, then, on the treated harvest, said treatment consisting in clarification with or without a prior HPH step. Results are presented in Table 5 in the form of percentages to be compared to the control value 100% representing the total HA amount present in the starting material, i.e. present in the untreated viral harvest.

TABLE 5
Effect of a prior HPH step on HA yield after clarification

| | | Clarification | Prior High-pressure | HA |
|---|---|---|---|---|
| | Pre- | -Type | Homogenization | yield |
| Experiments | clarification | of filter- | (HPH) | (%) |
| WiP136 | − | C0HC | − | 65 |
| (A Wisconsin | | | + | 85 |
| strain) | | | | |
| WiP137 | + | C0HC | − | 27 |
| (A Wisconsin | | | + | 53 |
| strain) | | | | |
| WiP137bis | + | C0HC | − | 18 |
| (A Wisconsin | | | + | 65 |
| strain) | | | | |
| MaP141 | + | Preflow | − | 84 |
| (B Malaysia | | | + | 135 |
| strain) | | | | |

Results—Conclusions

The results presented in Table 5 clearly indicate that the clarification step yield is increased if high-pressure homogenization is implemented before said clarification, irrespective of the type of filter used for clarification and for both A and B strains. Indeed, while it can be observed that the COHC filter, in the absence of homogenization, provides a lower yield than the Preflow filter, it is nonetheless noteworthy that performing a high-pressure homogenization before COHC filtration allows to get a virus yield up to 3.6 times higher. Interestingly, the HPH-induced HA yield increase is observed whether the virus harvest is left untreated (WiP136) or pre-clarified before HPH (WiP137, WiP137bis and MaP141).

### Example IV

### Effect of High-Pressure Homogenization of Virus-Containing Cell Culture Medium on Aggregation

In order to check how efficient the HPH step was in terms of decreasing aggregation present within the viral harvest, the CPS disc centrifuge, a particle size analyser, was used to analyse samples treated by HPH. The analyzer measures particle size distributions using centrifugal sedimentation within an optically clear spinning disc. The sample to be assessed migrates on a continuous sucrose gradient (6% to 18%) at a rotation speed of 18000 rpm. The particles in the sample begin to sediment as a thin band. If all particles are of the same size, they are settled at the same speed and arrive at the detector beam as a thin band. The time needed to reach the detector is used to calculate the size of particles. The distribution of particles size within a sample may be represented as a graph which is drawn by differential mode, i.e. the graph shows the quantity of material of each size, and the distribution is displayed as an absorption distribution (light absorbed/scattered plotted against particle size diameter). A solution of a purified influenza virus, known in the literature to have a spheric particle diameter of around 80 nm, was used as positive control, in order to get a reliable size control for the population of non-aggregated virus expected after homogenization.

### Effect of a 700 Bars Pressure

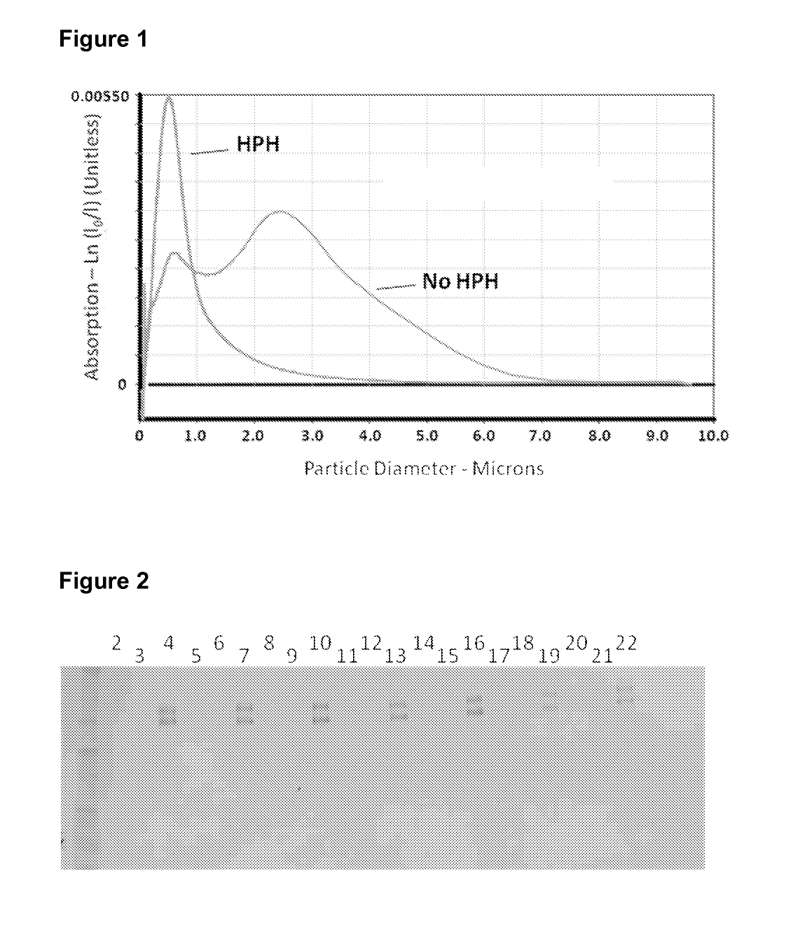

Cell culture and virus infection with a B Malaysia strain were as described in section I.1. of Example I. After collection, the virus-containing cell culture medium was subjected to high-pressure homogenization using the GEA Niro Soavi Panther™ system at a pressure of 700 bars and a flow rate of 100 L/h. A sample of said virus-containing cell culture medium was taken before and after homogenization and subjected to a CPS disc centrifuge analysis, as described above. Results are presented in FIG. 1.

Results—Conclusions:

As indicated on the graph displayed in FIG. 1, on the curve corresponding to the negative control with no HPH (“no HPH” curve), two populations were represented: (i) a first peak of particles having a diameter size between 0 and 2 μm, and (ii) a second peak of larger aggregates having a diameter from 2 to 6 μm. The curve corresponding to the homogenized sample (“HPH” curve, 700 bars) indicates a strong reduction of the population (ii) with a concomitant increase of the population (i), confirming that HPH induces dispersion of aggregation.

### Example V

### Homogenization of Influenza Virus-Containing Cell Culture Medium does not Affect the Virus Integrity

In order to demonstrate that virus integrity was maintained through homogenization, different analysis were performed on influenza virus-containing cell culture medium previously treated by high-pressure homogenization. Structural integrity of influenza virus was assessed by a sucrose cushion analysis and an electron microscopy analysis, while its infectivity was monitored by measuring the virus titration.

V.1. Sucrose Cushion Analysis

B/Malaysia strain was produced on MDCK cells essentially as described in the section I.1. of example I. After harvesting the virus by collecting the virus-containing cell supernatant, the viral harvest was subjected to high pressure homogenization using two different devices at varying pressures: 700 and 1000 bars with the Rannie™ homogenizer, and 700, 1000, 1250 and 1500 bars with the Panda™ homogenizer. After each homogenization, a sample of the homogenized viral harvest was laid carefully onto a 30% sucrose solution tube and then run for 3 hours at 30000 rpm at 4° C. in a Beckman 50.4 Ti rotor. After centrifugation, the tube was collected in 3 fractions:
1 ml collected from the top of the tube was called fraction S for Supernatant
1 ml was then collected and called fraction M for Middle (intermediate layer)
The rest of the tube was mixed with the pellet and noted as P for Pellet.

These 3 fractions were then analysed by Western-blot revealed with an anti-HA antibody. If the virus was lysed, i.e. its integrity has been altered, the virus bands should be detected in the 3 fractions. On the contrary, if the virus was recovered in an intact form, i.e. its integrity has been maintained, the virus bands should be detected only in the Pellet fractions. The Western-blot analysis is presented in FIG. 2.

Results—Conclusions

As clearly shown in FIG. 2, regardless of the pressure value used for homogenization (from 700 bars to 1500 bars), the HA signal was only recovered in the Pellet fraction of the sucrose cushion centrifugation, indicating that a high-pressure homogenization treatment has no negative effect on virus integrity.

### V.2. Electron Microscopy Analysis

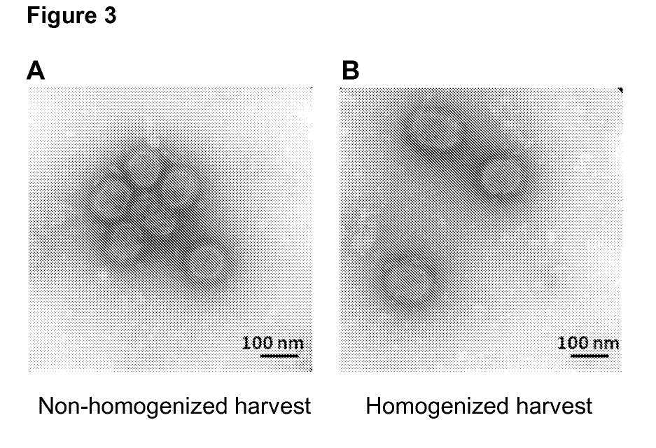

As a distinct analysis to assess virus integrity, the influenza virus-containing cell culture supernatants homogenized (FIG. 3A), as described in the experiment of Example IV, with a pressure of 700 bars, were examined by electron microscopy and compared with corresponding supernatants examined before homogenization (FIG. 3B). Representative pictures are presented in FIG. 3.

Results—Conclusion

FIG. 3 displays viruses visible beside cell debris. Viruses are aggregated before homogenization (FIG. 3A) and in an individual form after homogenization (FIG. 3B). In both conditions, viruses are intact and undistinguishable, indicating that homogenization by HPH does not affect virus integrity.

### V.3. Virus Titration

Virus viability was assessed by measuring the viral titration through the determination of the Tissue Culture Infectious Dose (TCID₅₀/ml) which represents the amount of a virus capable of infecting 50% of cells. Confluent MDCK cells cultured in 96 wells microplates were infected with serial dilutions of every influenza virus-containing samples differently homogenized according to the experiment described in the above section V.1. for 1 hour at 37° C. After elimination of the virus solution, medium was added and cells were kept at 35° C. for 5-7 days. The cells were then examined microscopically and scored as infected or not by monitoring the cytopathic effect (CPE) in cells. A suspension of infectious virus is used as a positive control to demonstrate cellular susceptibility and non-inoculated cultures are used as negative control. The number of wells where CPE was detected was scored for each dilution as infected cells and the viral titer was calculated according to Reed and Muench method (Reed, L. J. and Muench, H., 1938, The American Journal of Hygiene 27: 493-497). Titration results are presented in Table 6 and expressed as log TCID₅₀/ml.

TABLE 6
Effect of homogenization on virus titration

| | | | Viral Titer | |
|---|---|---|---|---|
| | Homogenization | | log TCID₅₀/ml | |
| Experiment | condition | | Before HPH | After HPH |
| MAP148 | Rannie ™ 700 | bars | 9.59 | 9.13 |
| | Rannie ™ 1000 | bars | 9.59 | 8.93 |
| | Panda ™ 700 | bars | 9.59 | 9.4 |
| | Panda ™ 1000 | bars | 9.59 | 9.4 |
| | Panda ™ 1250 | bars | 9.59 | 8.99 |
| | Panda ™ 1500 | bars | 9.59 | 9.54 |

Results—Conclusions

Table 6 indicates that influenza virus maintains its infectivity through high-pressure homogenization, as this step, regardless of the pressure value, does not significantly reduce the viral titers.

### Example VI

### Effect of a Prior Homogenization Step on Subsequent Filterability of the Virus Harvest

A consequence of decreasing the number and/or size of large aggregates present in the virus harvest to be clarified is to improve its filterability. In particular, diminishing aggregation will prevent, at least strongly limit, the plugging of filters. One advantage provided by this effect is to reduce the filter surface area required to clarify a given amount of virus harvest. Filterability of a suspension may be evaluated by two measures: (i) the inlet pressure measured during the filtration, and (ii) the surface area which is required to filter said suspension, that is, the higher the filterability is, the smaller the filtration surface area required is.

### VI. 1. Effect on Inlet Pressure

During the experiments WiP137bis and MaP141 (see Example III for their description), the pressure inlet was monitored during the filtration-based clarification step. Also, the final pressure inlet value was recorded, i.e. the value reached when the filtration was stopped, either because the totality of the volume was filtered (prior HPH step), or because the maximum limit of pressure, 20 psi, was reached before the totality of the volume could be filtered (no prior HPH step). As the experiments WiP137bis and MaP141 were performed in duplicates, one duplicate having no prior homogenization step before the indicated clarification step and one duplicate having a prior homogenization step before the same indicated clarification step, a comparison of the inlet pressure reached when clarifying, with or without a prior homogenization step, can be made. Results are presented in Table 7.

TABLE 7
Effect of a prior HPH step on inlet pressure during clarification
by filtration and on the filtration surface area

| | | Prior High-pressure | Final | Volume | Filter | Extrapolated |
|---|---|---|---|---|---|---|
| | Clarification - | Homogenization | pressure | filtered | area | Area |
| Experiment | Type of filter- | (HPH) | (psi) | (ml) | (cm²) | (m²/m³) |
| WiP137bis | C0HC | − | 20 | 195** | 23 | 12 |
| | | + | 8 | 496* | 23 | 5 |
| MaP141 | Preflow | − | 20 | 65** | 13.6 | 21 |
| | | + | 10 | 192* | 13.6 | 7 |

*Represents the totality of the volume, as the pressure limit was not reached when a prior HPH step was implemented
**Represents the volume which was filtered before the pressure limit, 20 psi, was reached

Results—Conclusions

Table 7 shows that the final pressure inlet is systematically lower when high-pressure homogenization is included in the process, from 2 to 2.5 times lower, indicating a higher filterability of the sample after homogenization.

### VI. 2. Effect on Filtration Surface Area

To determine whether the drop in pressure observed above has an impact on the filtration surface area required for clarifying a given volume, an extrapolation calculation has been made according to the following principle. If a filter with a surface of x cm² can process a volume of y ml before it is plugged or before the maximal inlet pressure is reached, then, it is possible to determine by extrapolation what filter surface in m² will be required per m³ of a viral-containing fluid to be clarified, using the following formula x/0.01y. The two next-to-last columns of the Table indicate the volume which was filtered (y) and the surface area of the filter (x), respectively. The last column of the Table indicates the extrapolated filtration surface area which was calculated according to the above principle

Results—Conclusions

The last column of Table 7 indicates that implementing a homogenization step allows to significantly reduce the surface filtration area required for clarification by an at least 2.4-fold factor and up to a 3-fold factor, compared to the surface required when no prior homogenization was implemented. This reduction was correlated with the pressure drop.

1. (canceled)

2. A method of purifying a virus, or a viral antigen thereof, from a culture of infected cells comprising at least the steps of:
(a) collecting the virus-containing cell culture medium, and
(b) purifying the virus,
wherein at least one homogenization step is implemented during the purification to obtain a virus homogenate.

3. (canceled)

4. The method according to claim 2, wherein one homogenization step is performed on the virus-containing cell culture medium.

5. The method according to claim 2, wherein the virus homogenate is further clarified.

6. (canceled)

7. The method according to claim 2, wherein step (b) comprises at least one purification step selected from the group of: clarification, nucleic acid degradation, ultrafiltration and sucrose gradient ultracentrifugation, or any combination thereof.

8. The method according to claim 7, wherein at least one clarification step is implemented and at least one homogenization step is performed before the clarification.

9. (canceled)

10. The method according to claim 7, wherein at least one ultrafiltration step is implemented and at least one homogenization step is performed after the ultrafiltration.

11. The method according to claim 7, wherein at least one sucrose gradient ultracentrifugation step is implemented and at least one homogenization step is performed before the sucrose gradient ultracentrifugation.

12. The method according to claim 7, wherein at least one clarification step, at least one ultrafiltration step, and at least one sucrose gradient ultracentrifugation step are implemented.

13. The method according to claim 12, wherein at least two homogenization steps are implemented.

14. The method according to claim 13, wherein at least one homogenization step is implemented before clarification and at least one homogenization step is implemented before the sucrose gradient ultracentrifugation.

15. The method according to claim 12, wherein the steps of homogenizing the virus-containing cell culture medium collected in step a, clarification, ultrafiltration, and sucrose gradient ultracentrifugation are implemented successively, and wherein the method further comprises an optional homogenization step between the steps of ultrafiltration and sucrose gradient ultracentrifugation.

16. The method according to claim 5, wherein clarification is performed by filtration or centrifugation.

17. (canceled)

18. The method according to claim 2, wherein the virus-containing cell culture medium is pre-clarified before being subjected to homogenization.

19. The method according to claim 2, wherein homogenization is performed by high pressure homogenization, and wherein the pressure ranges from 100-1500 bars.

20. The method according to claim 2, wherein homogenization is performed by sonication.

21. (canceled)

22. The method according to claim 2, further comprising a splitting step, at least one virus inactivation step, or both.

23. (canceled)

24. The method according to claim 2, wherein the cells are mammalian cells or avian cells.

25. The method according to claim 24, wherein the cells are mammalian MDCK cells or avian EB66® cells.

26. (canceled)

27. (canceled)

28. The method according to claim 2, wherein the virus is influenza virus.

29. (canceled)

30. An immunogenic composition comprising a virus, or a viral antigen thereof, obtained according to claim 2 admixed with a suitable pharmaceutical carrier.

31. A method for the preparation of a vaccine comprising at least the step of admixing the virus obtained according to claim 2 with a pharmaceutically acceptable carrier.
